(19) United States
(12) Patent Application Publication
(10) Pub. No.: US 2008/0242718 A1
(43) Pub. Date: Oct. 2, 2008

(54) System and method for using platelet biomarkers for diagnosis and treatment of neuro-psychiatric conditions

(76) Inventor: Sanjay Jasuja, Menlo Park, CA (US)

Correspondence Address: Sanjay Jasuja, P.O.Box 7829, Menlo Park, CA, 94026, US

(21) Appl. No.: 12/075,947

(22) Filed: Mar. 14, 2008

Related U.S. Application Data
(60) Provisional application No. 60/906,913, filed on Mar. 14, 2007.

(51) Int. Cl. A61K 31/404; C12Q 1/02; A61K 31/135; G06Q 50/00; G06Q 10/00; A61P 25/00; C12M 1/34

(52) U.S. Cl. 514/415; 435/29; 435/287.1; 514/654; 705/2

(57) ABSTRACT

The invention concerns in-vitro evaluation of intracellular biomarkers by doing blood testing. Concentrations of neurotransmitters in the platelet cells are utilized to identify chemical imbalance in the central nervous system. In one embodiment of the invention, testing data is analyzed by proprietary software algorithms by physicians and related health care professionals to diagnose and treat sufferers of chemical imbalance objectively. In another embodiment of the invention the testing and software predetermines risks of developing various neuropsychiatric conditions, like depression, Parkinson's, Alzheimer's, bipolar disorder, ADD/ADHD, autism, asperger's syndrome, OCD and other anxiety disorders, muscle disorders, etc.

### CROSS-REFERENCE TO RELATED APPLICATIONS

This application claims the priority benefit of U.S. provisional patent application 60/906,913, “In-Vitro diagnostic evaluation of proteins, amines, peptides associated with mental and neurological disorders, and analyzing it by software for diagnosis, treatment, prognosis and predetermination of risks,” filed on Mar. 15, 2007. The contents of this application are incorporated herein by reference.

### BACKGROUND OF THE INVENTION

Neurotransmitters are chemicals involved in the transmission of messages from one neuron to the other. They are biogenic amines, monoamines, amino acids, peptides, etc. In the monoamine group we have serotonin, histamine, acetylcholine, nor-epinephrine, epinephrine, Dopamine, etc. Some of the other neurotransmitters are glutamate, gamma amino butyric acid, etc.

It is estimated up to ten to fifteen percent of the global population is affected by some form of neurotransmitter imbalance. This is a major public health issue, and ideally rapid and objective means of treating these disorders would be highly desirable. Unfortunately, however, current medical methods of diagnosing and treating these chemical imbalances by actual measurements are in a relatively primitive state.

Rather than objective clinical tests, at present, most neuro-psychiatric disorders are diagnosed by a physician's subjective evaluation of a patient's case history. For example, at present, diagnosis of neuro-psychiatric disorders like depression, bipolar disorder, anxiety disorders, Parkinson's, A.D.H.D., etc. is made by subjective assessments. The physician typically asks questions, such as: “Are you feeling down?” “Have you lost interest in activities you liked?”; “Are you thinking of taking your life?”; “Are you hearing voices?,” etc. Depending upon the physician's subjective assessment, if for example the physician believes that the patient may be depressed, he or she will prescribe psychotropic medications. The patient will then try the medications, and a number of weeks later, the physician will then schedule follow up visits to determine if the medication is working.

The process is thus one of trial and error, and not infrequently, the patient may have to try several medications, in search of the right one. During this trial and error process, various side effects may occur because some of these drugs may induce their own neurotransmitter imbalances. Patients often end up feeling as if they are experimental guinea pigs, and unfortunately often their suffering continues during this trial and error process. An additional problem is that the accompanying side effects and failure of treatment can leads to non-compliance. Patients can stop taking their medications, lose trust in their doctor and treatment, and continue to deteriorate.

As a result, objective diagnostic tests and treatment methods, and equipment that could more rapidly and objectively determine a patient's true neuro-psychiatric status and easily identify correct treatment, without ‘trial and error,’ would be highly beneficial. Such methods could reduce patient suffering, improve compliance, and reduce medical costs.

The invention is novel and revolutionary. It changes the way neuropsychiatric conditions are diagnosed and treated

### BRIEF DESCRIPTION OF THE INVENTION

Surprisingly, it has been experimentally found that for a number of psychiatric disorders, there is a good correlation between the levels of various neurotransmitters in platelets, and patient neuro-psychiatric status. Platelet levels are obtained using standard blood sampling techniques, and their chemical composition determined by clinical laboratory assays. An additional advantage of platelets is that their chemical composition tends to be relatively unaffected due to daily fluctuations, physical or psychological stress.

Based on these experimental findings, the present invention consists of a system and method for using platelet neurotransmitter assays to help guide patient neuro-psychiatric diagnosis, and help select and adjust appropriate treatment regimens.

In one embodiment, the invention consists of computer or computerized device software that recommends particular platelet assays based on an initial tentative patient neuro-psychiatric impression, refines the patient neuro-psychiatric diagnosis based upon the results of the platelet assays, and may additionally make recommendations as to what drug treatment may be more suitable for the situation at hand.

In other embodiments, the invention consists of a slide rule device that can also do these functions.

In still other embodiments, the invention consists of a point-of-care platelet analyzer device optimized for neuro-psychiatric analytes.

In still other embodiments, the invention consists of a method of treating neuro-psychiatric disorders using platelet neurotransmitter information and the algorithms discussed herein.

### BRIEF DESCRIPTION OF THE DRAWINGS

FIG. 1A, 1B, algorithm-low Dopamine

FIG. 2A, 2B, 2C, algorithm-low Serotonin

FIG. 3A, 3B algorithm-high Serotonin

FIG. 4A, 4B, algorithm-high Dopamine

FIG. 5A, 5B, algorithm-nor-epinephrine imbalances

FIG. 6A, 6B, algorithm-M A O enzyme imbalance

FIG. 7A, 7B algorithm-epinephrine imbalances

FIG. 8, algorithm-Metanephrine and Nor-Metanephrine imbalances

FIG. 9, algorithm-Tyramine imbalance

FIG. 10: To illustrate claim number four

FIG. 11: To illustrate claim number seven

FIGS. 12 and 13: To illustrate claim number eight

FIG. 14: To illustrate claim number ten

FIG. 15: To illustrate claim number eleven

Charts 1-18 showing improvement with use of present invention

### DETAILED DESCRIPTION

One aspect of the present invention is to utilize levels of neurotransmitters in platelet cells, because these levels are reliable as compared to plasma levels, feed the data to propriety software algorithms for ascertaining chemical imbalance in the central nervous system and use it to objectively diagnose and treat neuropsychiatric conditions, including prediction of risk factors.

One embodiment, the algorithms of the present invention may be implemented into software intended to run on a computerized device, such as a personal computer, laptop computer, hand held computer, or other device equipped with memory, input means, and display output means. In this case the invention will accept the patient status, weight, and platelet neurotransmitter levels as input, and make recommendations based on the described algorithms as output.

Using algorithms, 901 test samples were done for cases that came to the ADHD clinic. Neurotransmitter levels in platelet cells were studied. Out of 901, 257 were for dopamine, 256 for norepinephrine, 257 for serotonin and 131 for MAO (Mono Amine Oxidase) enzyme. Out of 257 dopamine samples, 178 had Dopamine imbalances. 156 had low Dopamine (DA) levels and 22 high Dopamine levels. Only 79 had normal dopamine. Out of 256 norepinephrine samples, 170 had nor-epinephrine (NE) imbalances. Out of this 136 had low levels of nor-epinephrine, 34 high levels and only 86 were normal. Out of 257 serotonin samples, 203 had serotonin (5HT) imbalances. Out of this, 203 had low levels, none had high level and only 54 were normal. The results of platelet levels of dopamine, norepinephrine and serotonin are recorded on chart 1, MAO enzyme on chart 2, dopamine only on chart 3, serotonin only on chart 4 and norepinephrine on chart 5. Reference range of normal levels is described in the ‘Methods’ section.

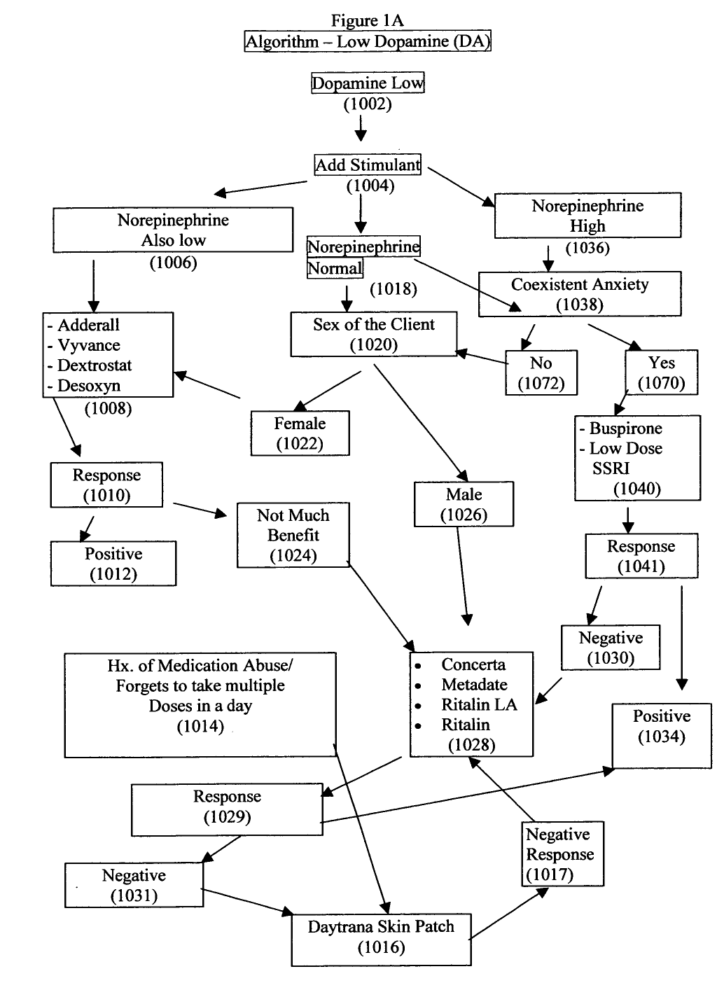

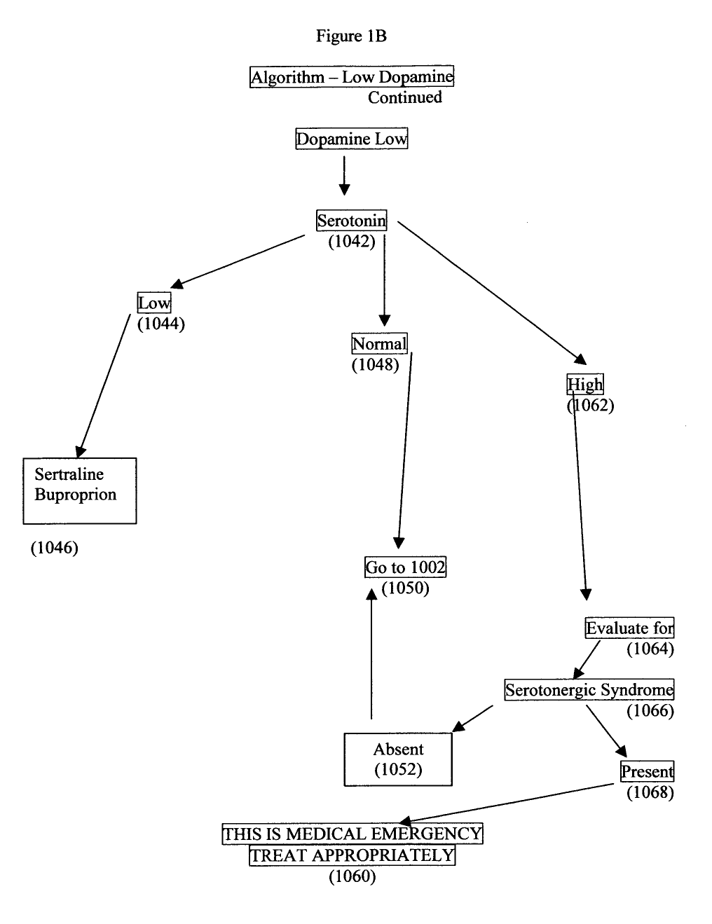

Algorithm FIG. 1A, 1B illustrates an exemplary system of the present invention.

### Algorithm, FIG. 1A, 1B, Low Dopamine

If just dopamine is low, add a stimulant drug, as in section 1004 of the algorithm. If Nor-epinephrine is normal, and dopamine is low, in male patients use the drugs in section 1028. In females, first try drugs in section 1008. In our experience these may have better effects. As in section 1036 and 1038, if Nor-epinephrine is high and there is coexistence anxiety, consider using a drug in section 1040, along with stimulant drug. If female patients do not respond well to a drug in section 1008, try a drug from section 1028. As mentioned in section 1014, if there is history of self-medication or substance abuse, or patient forgets to take multiple doses in a day, use the drug in section 1016. If that does not work, document it and try drugs in section 1028, the long-acting ones.

If along with Dopamine, Serotonin is also low, as in section 1044, then use drugs in section 1046. If Serotonin is high, evaluate the client for serotonergic syndrome. Common signs of serotonergic syndrome are high-temperature, muscle rigidity, diaphoresis, increased heart rate, agitation, etc. As in section 1060, this is a medical emergency, which could be lethal.

Using this algorithm, 257 test samples were evaluated out of the total 901 test samples. Neurotransmitter levels in platelet cells were studied. As illustrated in chart 3, out of 257 dopamine samples, 178 had Dopamine imbalances. 156 of these had low Dopamine (DA) levels and 22 high Dopamine levels. Only 79 were normal.

Some physicians like to treat patients struggling with neuropsychiatric disorders with few medications they refer to as their ‘favorite drugs.’For example, a patient with depression and anxiety is given an SSRI (selective serotonin reuptake inhibitor), even though there may be coexistent nor-epinephrine and or Dopamine imbalances.

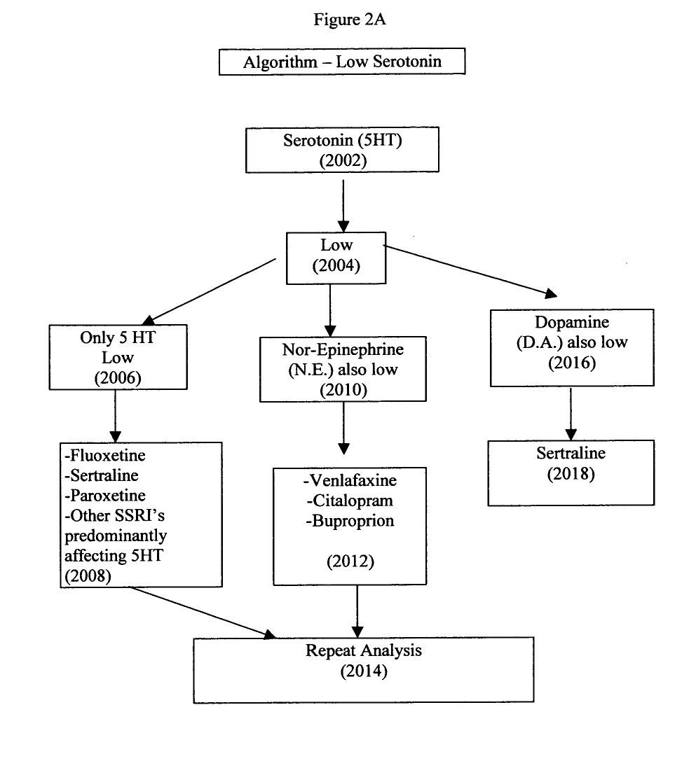

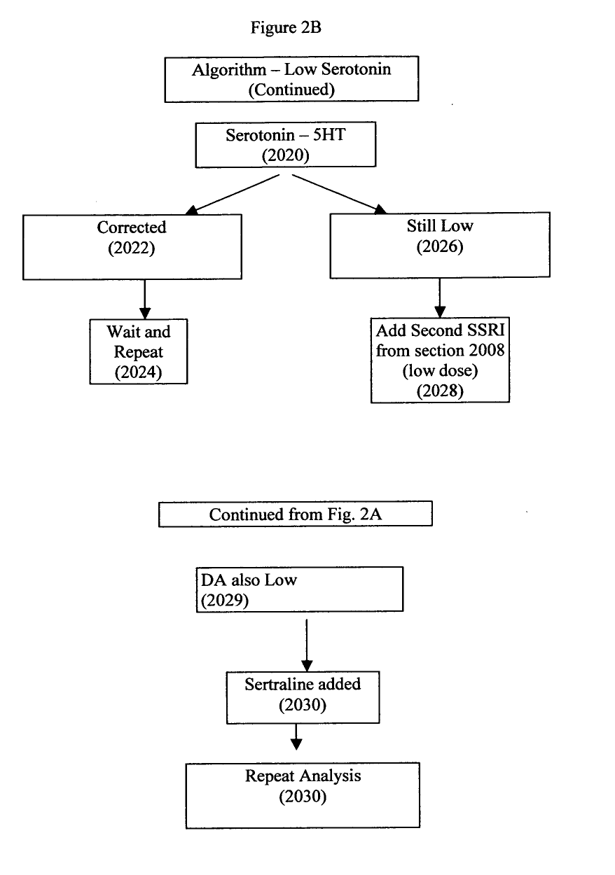

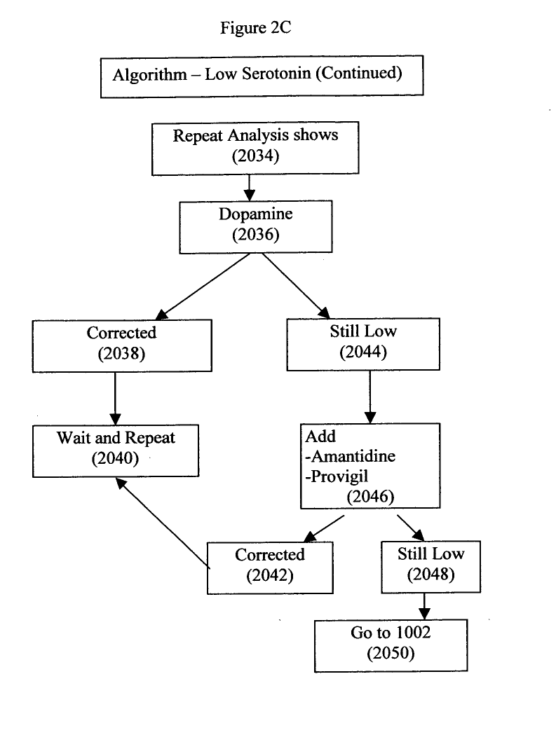

According to the present invention, exact brain chemical imbalance is identified in each case and the software guides the physician to use only the drug(s) that will correct it.
FIG. 2 illustrates another exemplary system of the present invention.

### Algorithm, FIG. 2A, 2B, Low Serotonin:

If platelet neurotransmitters levels show that serotonin is low and Nor-epinephrine and Dopamine are normal, proceed to Section 2006 and try drugs in section 2008. If response is positive, repeat the analysis in three months.

Besides low Serotonin, if Nor-epinephrine is also low, as in Section 2010, try drugs in section 2012. If along with Serotonin, Dopamine is also low, as in Section 2016, try the drug in Section 2018 as it provides positive effect on Serotonin, as well as mild benefits on Dopamine. Upon repeat analysis, if Serotonin level is corrected, go to section 2024. If it is still low, as in Section 2026 consider adding low dose of another serotonergic drug mentioned in section 2008.

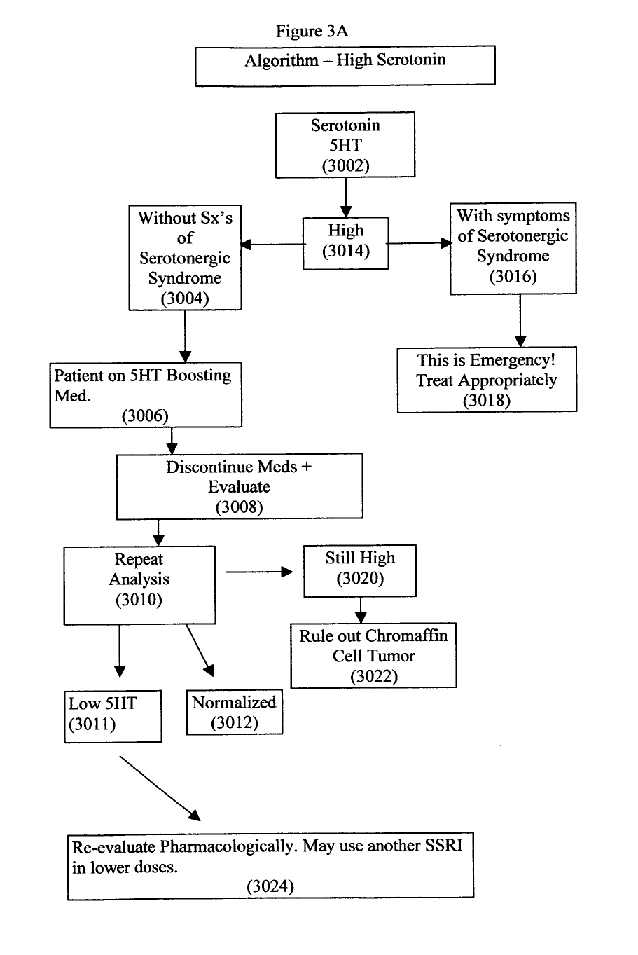

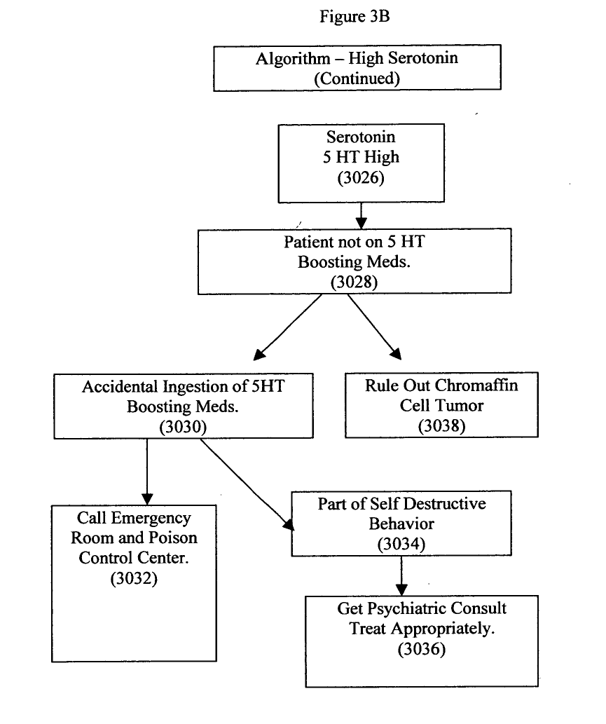

If serotonin is corrected and dopamine is still low, try drugs in section 2046 and repeat levels in three months. If Dopamine level is corrected this way, go to section 2042. If dopamine is still low, then consider drugs in Section 1002.
Imbalance verses balance pattern of Dopamine, Serotonin and Nor-epinephrine are depicted in FIGS. 3-5.

### Algorithm, FIG. 3A, 3B, High Serotonin:

If Serotonin level is high as in Section 3014, follow algorithm to section 3004, 3016, etc. If there are no symptoms of serotonergic syndrome, follow to section 3006 and 3008. If symptoms of serotonergic syndrome are present, go to Section 3018. This is a medical emergency.

If there are no symptoms of serotonergic syndrome and patient is on serotonin boosting medication, as in section 3006, go to section 3008 and discontinue serotonin boosting drug. If after discontinuation of the drug, repeat testing shows serotonin levels still high, as in section 3020, chromaffin cell tumor needs to be ruled out, as in section 3022. Make immediate referral to an endocrinologist. If after discontinuing serotonin-boosting drug, as in section 3008, repeat analysis shows lower serotonin levels, evaluate the patient and consider another SSRI (selective serotonin reuptake blocker) in lower doses, as in section 3024 and so on (see FIG. 3B).

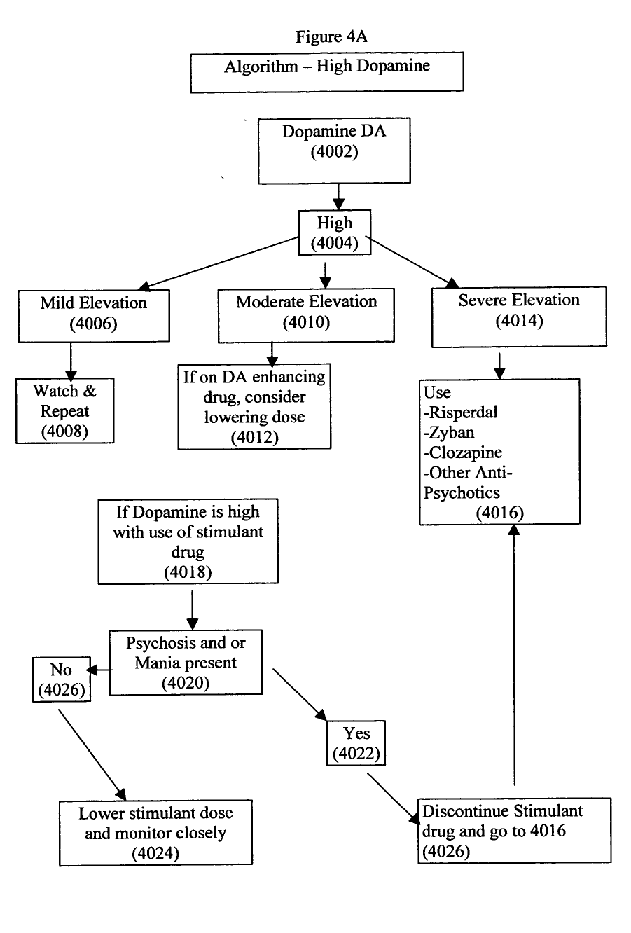

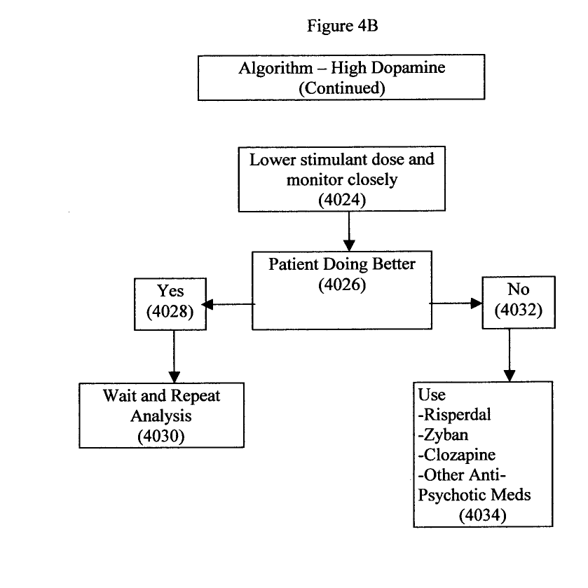

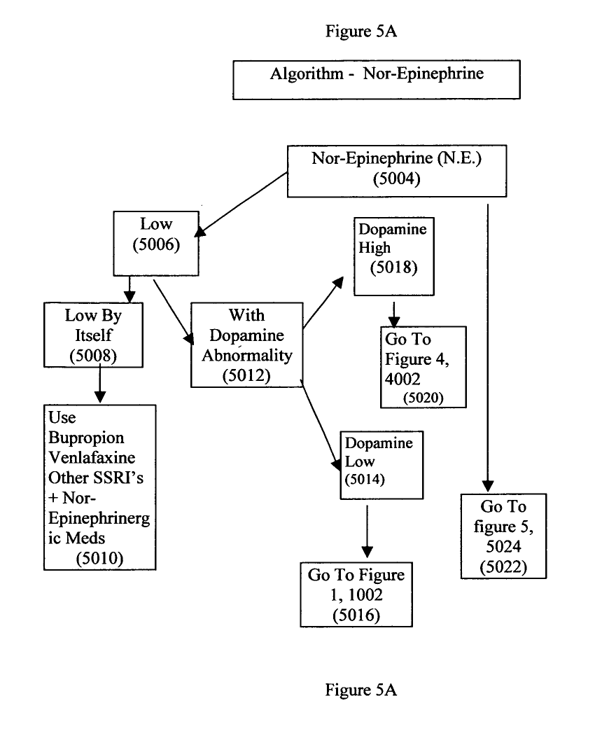

### Algorithm, FIG. 4A, 4B, High Dopamine:

If levels of platelet dopamine are high without any clinical symptoms, follow patient carefully and repeat them in three months, as in section 4008. If there is moderate elevation, as in section 4010 and if on dopamine boosting drug, consider lowering its dose as in section 4012. If the Dopamine level is very high, consider using drugs mentioned in section 4016. If patient is on stimulant drug (section 4018) and dopamine level is very high, with no psychosis and or mania, lower the dose as in Section 4024. If psychosis and or mania is present (section 4022), discontinue stimulant drug and use drugs in Section 4016.

As in Section 4024 (FIG. 4B), if you have already lowered the dose of stimulant drug and there is no considerable improvement, use drugs in Section 4034.

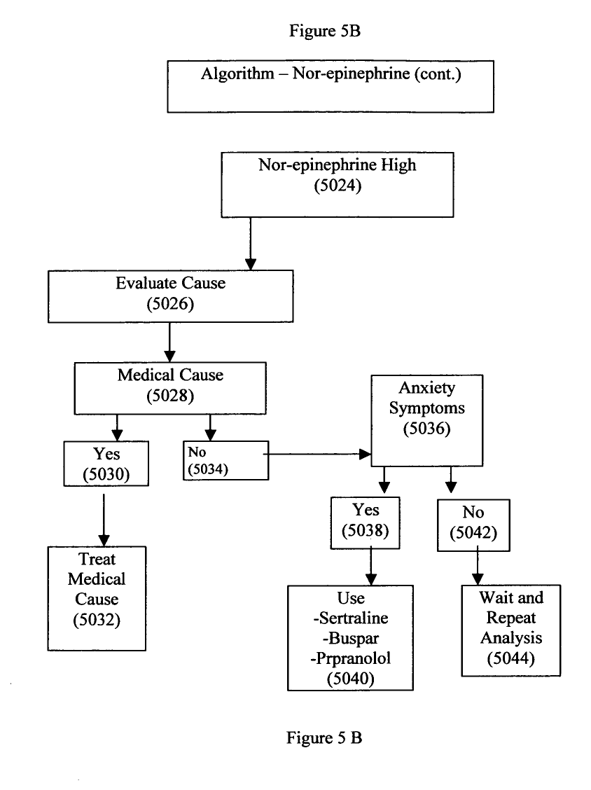

### Algorithm, FIG. 5A, 5B, Nor-Epinephrine:

If only nor-epinephrine is low, use drug in section 5010. If it is low along with a Dopamine abnormality, consider recommendations in Section 5014, 5018 and so on. If Nor-epinephrine is high, go to Section 5026 and evaluate a medical cause as in Section 5028. If medical cause is present, follow algorithm to Section 5030 and 5032. If there is no medical cause, and anxiety symptoms are present, use drugs in Section 5040. If there are no anxiety symptoms, follow recommendations in Section 5044.

Using these algorithms, as illustrated in chart 3 and 5, it is interesting to note that although number of samples of dopamine and norepinephrine imbalance were different, their graph patterns were similar. Most cases had low levels, smaller number of cases had high levels and the number of cases with normal levels was in the middle. In the serotonin graph, as illustrated in chart 4, it is notable that none of the cases showed high serotonin level. If they did, they would be in what is called, ‘serotonergic crisis,’ also called, ‘serotonin syndrome,’ or ‘serotonergic syndrome,’ which is an emergency situation needing immediate and urgent medical attention.

Test data from cases suspected of noncompliance or self-medication was excluded to maintain the purity of the data.

As an example of this algorithm, these methods were used for a 52-year-old female. Her Biomarker levels are illustrated in chart 6. In April '03, the levels of nor-epinephrine, dopamine and serotonin were 2.9, 19.3 and 48.2. In August '07, they were 2.4, 34.7 and 59.2 respectively. This was despite the fact that few months earlier in 2007, she was hospitalized with a brief episode of psychosis. She was off of the stimulant medication and on an anti-psychotic and mood stabilizer. She was sleepy and appeared ‘drugged,’ most of the day. Her husband and kid took over all the household responsibilities. Her attention span and alertness were ‘down in the dumps’.

She did not like her condition and became depressed. The fact her dopamine level was normal, decision was taken to start her on a different stimulant medication, low dose, with close monitoring. She responded well and was slowly taken off the anti-psychotic. Currently, she is on a stimulant, antidepressant and mood stabilizer. She is a well functioning adult and takes care of her household responsibilities. If we would not have used this platelet neurotransmitter technology and analyzed her platelet neurotransmitter levels, she would not have been put on stimulant medication for her ADHD and probably continued on the antipsychotic medication for the rest of her life. The technology has changed her life in a positive way.

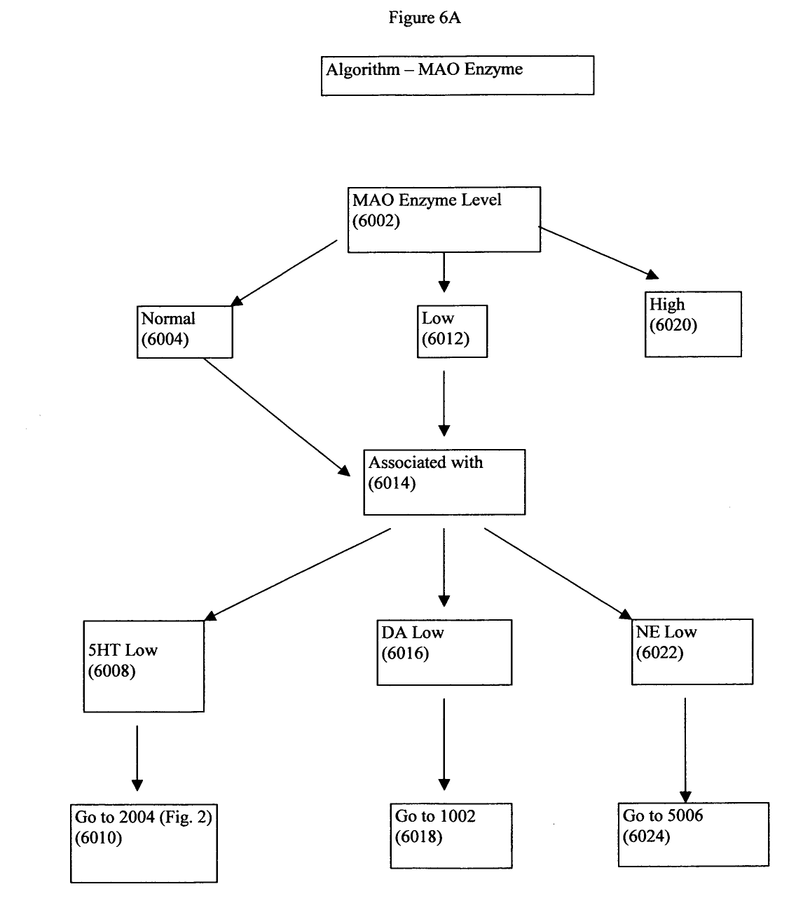

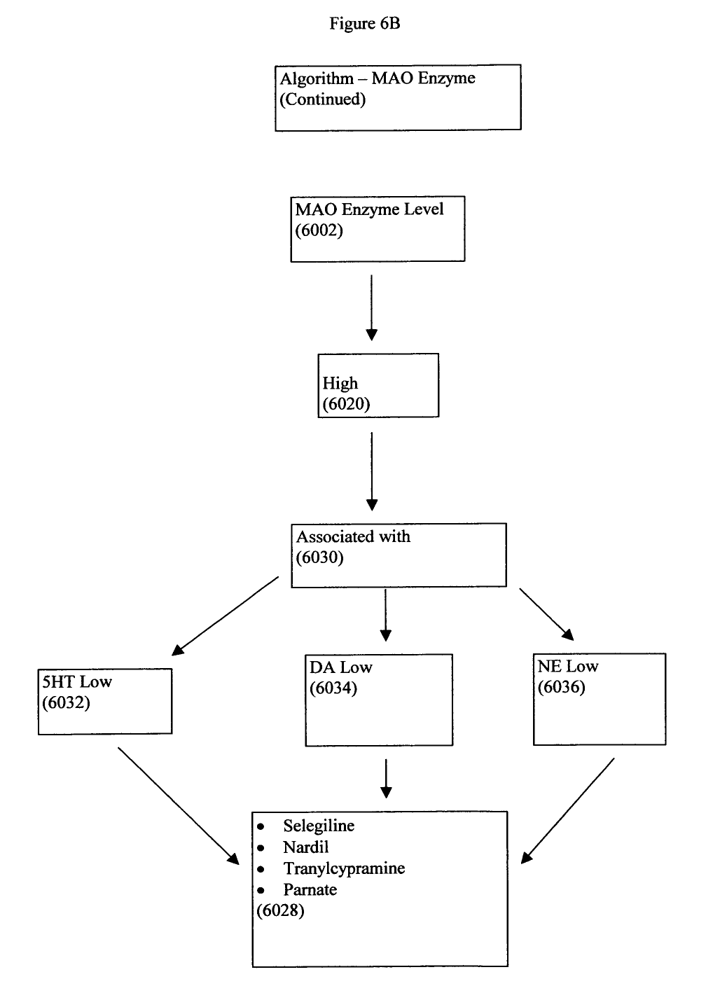

### Algorithm, FIG. 6A, 6B, MAO (Mono Amine Oxidase) Enzyme:

If MAO enzyme is normal or low and Serotonin is low, follow the algorithm to section 6010. If low MAO is associated with low Dopamine or low Nor-epinephrine, follow the algorithm to 6018 and 6024 respectively. If MAO level is high, as in Section 6020 and is associated with low Serotonin, Dopamine and or Nor-epinephrine, use drug in section 6028. These drugs are called MAOI or MAO Inhibitors, and should be used with special caution, after considering their side effects.

In 131 test samples out of 901, levels of MAO (nmol/h) were measured. As illustrated in chart 2, 54 were low, 74 were normal and only 3 had high MAO levels. This would mean only in 2.2% of the above samples, there could be a benefit of using an MAOI type of drugs, some of which are said to have mild stimulant properties. So this kind of testing and analysis enables personalized medicine. It objectifies the exact chemical imbalance(s) so it can be treated accordingly, without wasting time, money or resources.

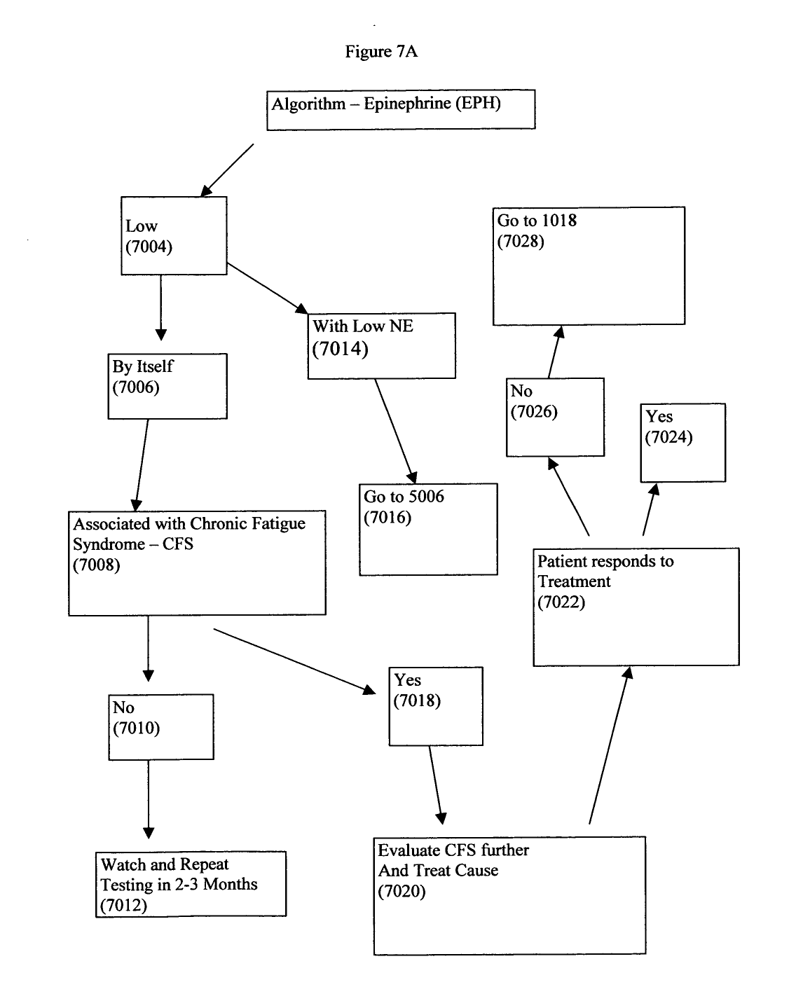

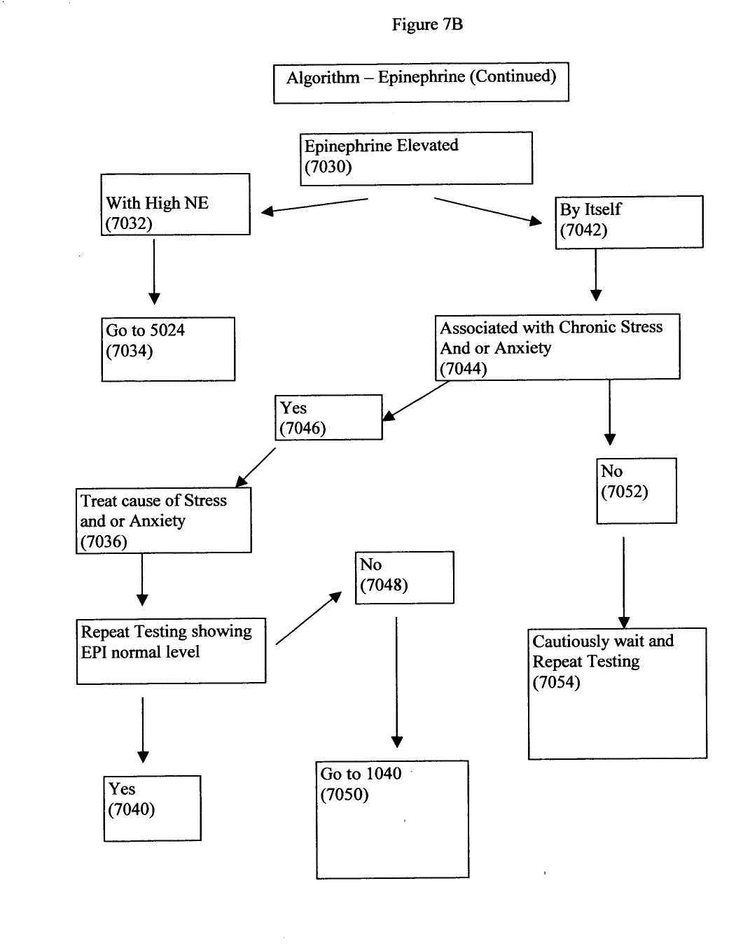

The algorithms for Epinephrine are shown in FIGS. 7A and 7B

### Algorithm, FIG. 7A, 7B Epinephrine:

If platelet epinephrine is low by itself, as in Section 7006, follow the algorithm all the way to Section 7012, 7020, 7022, etc. If it is associated with low nor-epinephrine, as in section 7014, go to section 5006.

If epinephrine is elevated along with nor-epinephrine, go to Section 5024. If epinephrine is elevated all by itself, as in Section 7042, follow the algorithm in Section 7044 all the way to Sections 7040, 7050 and 7054.

As an example of this algorithm, these methods were used for a 39-year-old divorced female. She had a history of extreme anger outbursts, attentional difficulties and high sexual drive. She had never been able to maintain a stable job. Had long been in psychotherapy and had a history of suicidal ideations from time to time. She would make impulsive decisions and get obsessed over them. Her neurotransmitter levels were as follows (see Table 1 below):

TABLE 1

| Test date | NE | DA | 5HT |
|---|---|---|---|
| December 1997 | 0.2 | 3.1 | 3.2 |
| April 2003 | 6.3 | 16.8 | 31.2 |
| November 2003 | 6.6 | 17.3 | 36.8 |
| August 2004 | 7.4 | 22.3 | 48.3 |
| August 2005 | 6.9 | 16.2 | 40.7 |

As illustrated in chart 7, with the use of technology of the present invention, her dopamine and serotonin levels were normalized with treatment. It is notable that it took about three years for dopamine and serotonin to get normalized, with a combination of ADHD medication and an antidepressant. Unfortunately, she discontinued treatment before her norepinephrine levels could be brought within the normal range (4-6).

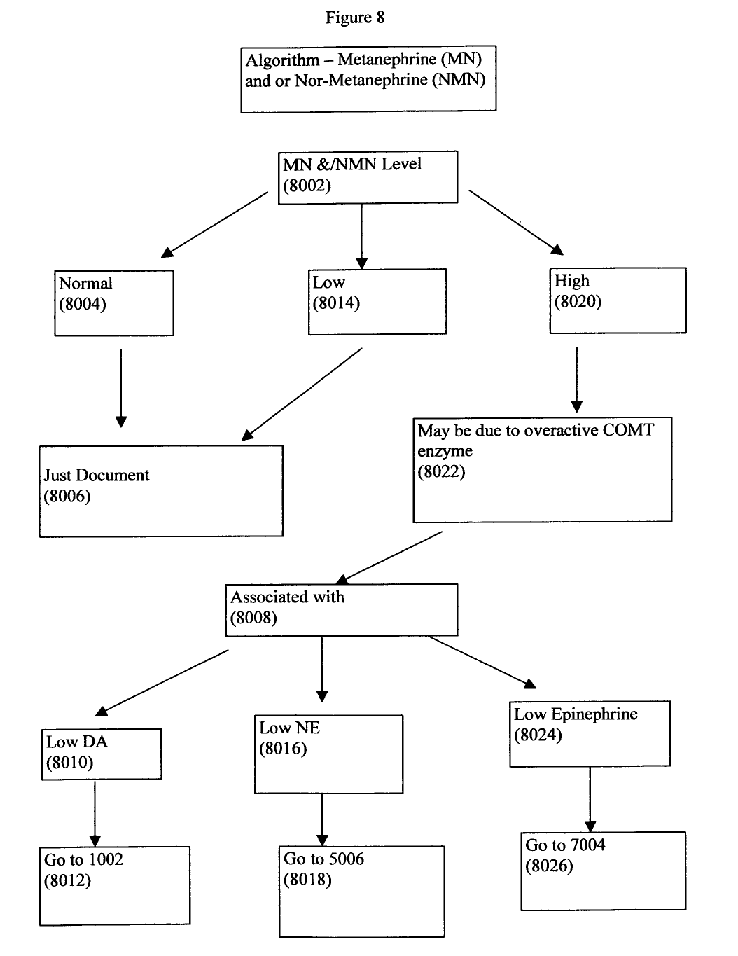

The algorithms for Metanephrine & Nor-Metanephrine are shown in FIG. 8.

### Algorithm, FIG. 8-Metanephrine and or Normetanephrine:

If these levels are normal or low, just document them. If they are high, as in Section 8020, follow the algorithm all the way to sections 8012, 8018 and 8026 respectively.

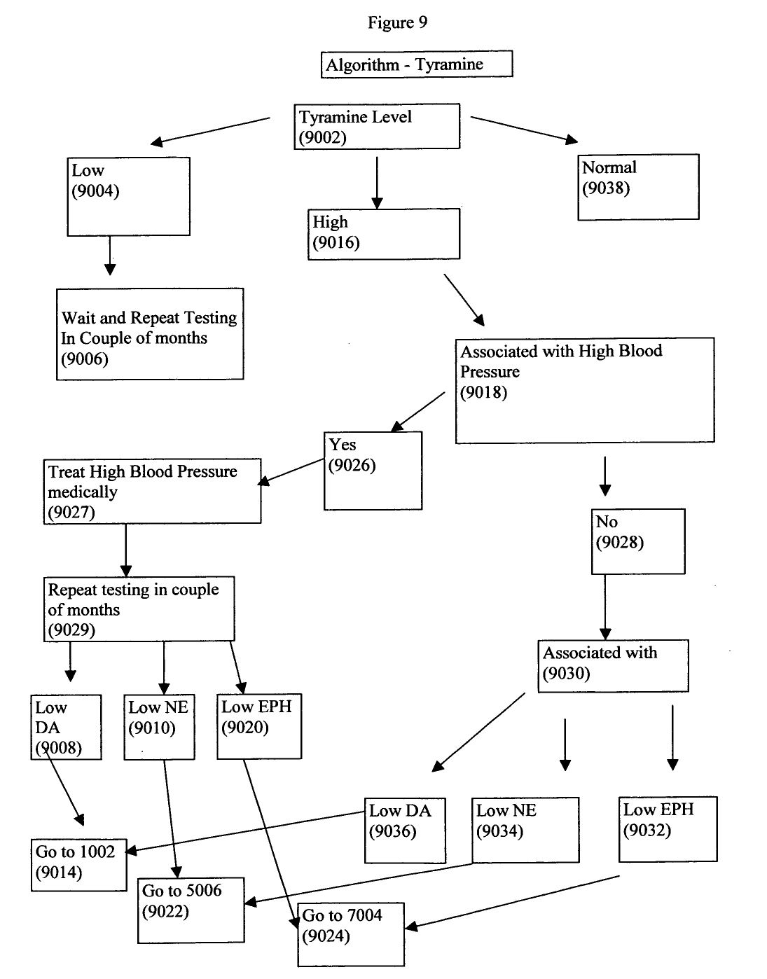

### Algorithm, FIG. 9, Tyramine:

If Tyramine is high, as in Section 9016, follow the algorithm to Section 9018 and so on. If it is low, go to Section 9006.

### Methods:

Patients were sent to a local lab. Non-fasting sample was collected by venepuncture, in sitting position, in 10 c.c. Lavender-top tubes. Platelet neurotransmitter concentrations were analyzed by research lab. Levels were reported as ng/10E10 platelets. Reference normal range was as follows: epinephrine 3 to 5, norepinephrine 4 to 6, Dopamine 17 to 30, Serotonin 30 to 400 for males and 65 to 550 for females, Metanephrine 4 to 22, Normetanephrine 4 to 30, Tyramine 4.4 to 16, Cortisol (ug %) 5 to 25, and MAO (nmol/h) 10.7 to 23.6 respectively. Treatment was decided according to the algorithms, all described together, (explained below):

Caution: During these therapeutic algorithms, patients should be followed closely by the prescribing physician.

### Algorithm, FIG. 1A, 1B Low Dopamine

If just dopamine is low, add a stimulant drug, as in section 1004 of the algorithm. If Nor-epinephrine is normal, and dopamine is low, in male patients use the drugs in section 1028. In females, first try drugs in section 1008. In our experience these may have better effects. As in section 1036 and 1038, if Nor-epinephrine is high and there is coexistence anxiety, consider using a drug in section 1040, along with stimulant drug. If female patients do not respond well to a drug in section 1008, try a drug from section 1028. As mentioned in section 1014, if there is history of self-medication or substance abuse, or patient forgets to take multiple doses in a day, use the drug in section 1016. If that does not work, document it and try drugs in section 1028, the long-acting ones.

If along with Dopamine, Serotonin is also low, as in section 1044, then use drugs in section 1046. If Serotonin is high, evaluate the client for serotonergic syndrome. Common signs of serotonergic syndrome are high-temperature, muscle rigidity, diaphoresis, increased heart rate, agitation, etc. As in section 1060, this is a medical emergency, which could be lethal.

### Algorithm, FIG. 2A, 2B, Low Serotonin:

If platelet neurotransmitters levels show that serotonin is low and Nor-epinephrine and Dopamine are normal, proceed to Section 2006 and try drugs in section 2008. If response is positive, repeat the analysis in three months.

Besides low Serotonin, if Nor-epinephrine is also low, as in Section 2010, try drugs in section 2012. If along with Serotonin, Dopamine is also low, as in Section 2016, try the drug in Section 2018 as it provides positive effect on Serotonin, as well as mild benefits on Dopamine. Upon repeat analysis, if Serotonin level is corrected, go to section 2024. If it is still low, as in Section 2026 consider adding low dose of another serotonergic drug mentioned in section 2008.

If serotonin is corrected and dopamine is still low, try drugs in section 2046 and repeat levels in three months. If Dopamine level is corrected this way, go to section 2042. If dopamine is still low, then consider drugs in Section 1002.

### Algorithm, FIG. 3A, 3B, High Serotonin:

If Serotonin level is high as in Section 3014, follow algorithm to section 3004, 3016, etc. If there are no symptoms of serotonergic syndrome, follow to section 3006 and 3008. If symptoms of serotonergic syndrome are present, go to Section 3018. This is a medical emergency.

If there are no symptoms of serotonergic syndrome and patient is on serotonin boosting medication, as in section 3006, go to section 3008 and discontinue serotonin boosting drug. If after discontinuation of the drug, repeat testing shows serotonin levels still high, as in section 3020, chromaffin cell tumor needs to be ruled out, as in section 3022. Make immediate referral to an endocrinologist. If after discontinuing serotonin-boosting drug, as in section 3008, repeat analysis shows lower serotonin levels, evaluate the patient and consider another SSRI (selective serotonin reuptake blocker) in lower doses, as in section 3024 and so on (see FIG. 3B).

### Algorithms FIG. 4A, 4B, High Dopamine:

If levels of platelet dopamine are high without any clinical symptoms, follow patient carefully and repeat them in three months, as in section 4008. If there is moderate elevation, as in section 4010 and if on dopamine boosting drug, consider lowering its dose as in section 4012. If the Dopamine level is very high, consider using drugs mentioned in section 4016. If patient is on stimulant drug (section 4018) and dopamine level is very high, with no psychosis and or mania, lower the dose as in Section 4024. If psychosis and or mania is present (section 4022), discontinue stimulant drug and use drugs in Section 4016.

As in Section 4024 (FIG. 4B), if you have already lowered the dose of stimulant drug and there is no considerable improvement, use drugs in Section 4034.

### Algorithm, FIG. 5A, 5B. Nor-Epinephrine:

If only nor-epinephrine is low, use drug in section 5010. If it is low along with a Dopamine abnormality, consider recommendations in Section 5014, 5018 and so on. If Nor-epinephrine is high, go to Section 5026 and evaluate a medical cause as in Section 5028. If medical cause is present, follow algorithm to Section 5030 and 5032. If there is no medical cause, and anxiety symptoms are present, use drugs in Section 5040. If there are no anxiety symptoms, follow recommendations in Section 5044.

Algorithm. FIG. 6A, 6B, MAO (Mono Amine Oxidase) Enzyme:

If MAO enzyme is normal or low and Serotonin is low, follow the algorithm to section 6010. If low MAO is associated with low Dopamine or low Nor-epinephrine, follow the algorithm to 6018 and 6024 respectively. If MAO level is high, as in Section 6020 and is associated with low Serotonin, Dopamine and or Nor-epinephrine, use drug in section 6028. These drugs are called MAOI or MAO Inhibitors, and should be used with special caution, after considering their side effects.

### Algorithm, FIG. 7A, 7B, Epinephrine:

If platelet epinephrine is low by itself, as in Section 7006, follow the algorithm all the way to Section 7012, 7020, 7022, etc. If it is associated with low nor-epinephrine, as in section 7014, go to section 5006.

If epinephrine is elevated along with nor-epinephrine, go to Section 5024. If epinephrine is elevated all by itself, as in Section 7042, follow the algorithm in Section 7044 all the way to Sections 7040, 7050 and 7054.

### Algorithm, FIG. 8-Metanephrine and or Normetanephrine:

If these levels are normal or low, just document them. If they are high, as in Section 8020, follow the algorithm all the way to sections 8012, 8018 and 8026 respectively.

### Algorithm, FIG. 9, Tyramine:

If Tyramine is high, as in Section 9016, follow the algorithm to Section 9018 and so on. If it is low, go to Section 9006.

As another case history illustrating the use of these algorithms, consider the use of this technology in a 10-year-old female. She had a history of abnormally high level of energy, impulsiveness and anger outbursts. Her biomarker levels are shown in Table 2 below:

TABLE 2

| Test date | NE | DA | 5HT |
|---|---|---|---|
| January 2002 | 5.7 | 18.6 | 16.3 |
| September 2002 | 5.8 | 19.2 | 29.3 |

As shown in chart 8a, her serotonin levels were raised with few months of treatment. In September '02 testing they were almost within normal limits (30-550). They went from 16.3 to 29.3, an improvement of about 81 percent. The anger outbursts were not there anymore and her school performance improved, along with impulsivity issues.

Chart 8b demonstrates the use of present invention in an 8-year-old boy who had been misdiagnosed with Pervasive development disorder (a domain of disorders that includes autism, Asperger's syndrome and autistic like conditions). He also had difficulties in paying attention, learning issues and poor social skills. His levels are shown in Table 3 below:

TABLE 3

| Test date | NE | DA | 5HT |
|---|---|---|---|
| December 1997 | 0.4 | 0.4 | 9.4 |
| May 1999 | 3.8 | 7.8 | 11.2 |
| May 2001 | 6.1 | 16.3 | 30.2 |

With the use of present invention correct diagnosis and treatment was clarified. His parents were much relieved to know that their only son was not autistic. He struggled with ADHD, anxiety and leaning disabilities. His norepinephrine levels were normalized by the second testing; dopamine and serotonin levels were normalized by the third testing. He was able to move from a private, special learning and physically disabled school to a regular public school. Here he started doing well with the help of resource room teacher.

As an example, consider the use of the present invention in a 12-year-old boy with a history of: difficulties with attention to detail and declining school performance. He was a successful athlete in school but the school performance was lowering his self-esteem. His biomarker levels are shown in Table 4 below:

TABLE 4

| Test date | NE | DA | 5HT |
|---|---|---|---|
| April 2002 | 2.1 | 18.3 | 14.7 |
| July 2002 | 4.4 | 18.6 | 22.5 |
| February 2003 | 3.8 | 21.6 | 23.3 |
| October 2003 | 4.2 | 20.8 | 26.6 |
| April 2004 | 4.0 | 21.2 | 30.4 |
| November 2004 | 4.4 | 24.6 | 25.9 |
| August 2006 | 4.2 | 28.3 | 38.7 |

He was put on a stimulant medication that has positive effect on the norepinephrine system, along with beneficial effects on the dopamine and serotonin too. As illustrated in chart 9, his norepinephrine levels were normalized within three months. Dopamine levels continued to improve, going from low normal to high normal, even though he stayed on the same medication and similar doses. It was very satisfying to see the continuous improvement in this boy over the course of several years. Seems like the correct medication triggered a positive change on Dopamine concentration which kept on getting better and better even though the medication dose remained unchanged. The reason his dose had not been increased was because the technology of this invention had shown by the second testing that norepinephrine and dopamine levels were on the rise. Even though the dose was not changed, serotonin levels normalized by the fourth testing and then continued to increase towards mid-normal. Using the current invention it was possible to just keep him on one drug and not add a serotonin boosting additional drug.

Using poly-pharmacy makes the pharmacological picture ‘murky’ and complicated. As illustrated in Table 4 above and chart 9, one of the embodiments of the invention to allow physicians to use as less number of drugs(s) as possible and get maximum benefit. Fewer drugs equates to less side effects for the patient. Objective blood testing and algorithms (FIG. 1 to 9) guide treatment, without using multiple drugs, just because a patient says so or the physician subjectively thinks so.

Another embodiment of the present invention is allows the use of lower doses, dictated by the severity of the chemical imbalance revealed by measurements of platelet neurotransmitters. Lower drug doses also lower possibility of side effects, leading to higher success of treatment and more compliant patients. All this boosts the doctor-patient relationship. The above case, depicted by levels in chart 9, starting seeing the physician less frequently for follow-ups because he kept on getting benefit from his fine-tuned, personalized, dose of the drug. Currently he is in college and proud of his accomplishments. Self-esteem is healthy and his parents are proud of him.

Another embodiment of the present invention is to benefit the economics of health care system. Fewer doctors' visits cuts down the cost of health care for the family and insurance carrier. Using less number of drugs and in low doses also saves costs for insurance companies.

Further double blind, placebo controlled studies need to be done to show economic benefits to healthcare programs, either private or sponsored by the government (like Medicaid, medi-cal, etc.). Even in countries like England, France, Spain, Canada, etc. where there is universal health care, lesser utilization of health services would cause less burden on the system and saves money for the agencies running such programs. The present invention is vital, particularly since the U.S. struggles to fix its health care system, characterized by overuse and polypharmacy.

Chart 10 illustrates the use of present invention in a 36-year-old man of clergy. He had a history of anger outbursts, failing on whatever job the archbishop would assign him, resulting in animosity towards the archbishop and blaming everyone else for his problems. As a child, he had extraordinary high level of energy and difficulties in sustaining attention to detail. He had been subject to ridicule by his peers and starting keeping himself aloof. His biomarker levels are depicted in Table 5 below:

TABLE 5

| Test date | NE | DA | 5HT |
|---|---|---|---|
| December 1997 | 0.5 | 1.0 | 3.0 |
| April 1999 | 4.8 | 11.3 | 18.4 |

On repeat testing, his norepinephrine levels had normalized. Dopamine and serotonin levels were so low to begin with that in about eighteen months they were still low. Increase in them had been significant, 500 percent and 18.75 percent, respectively. He was later transferred by the archdiocese to a Midwest location. Had he continued to use the technology of the present invention, it would have been interesting to see if continued treatment would have normalized dopamine and serotonin levels.

Chart 11 illustrates use of the present invention for a 9 year-old boy. He presented as a polite, obese youngster. Very creative and day dreamy. School performance had been declining due to inability to sustain attention. Self-esteem was low along with feelings of depression. His biomarker levels are depicted in Table 6 below:

TABLE 6

| Test date | NE | DA | 5HT |
|---|---|---|---|
| November 2000 | 4.1 | 11.4 | 16.9 |
| June 2001 | 4.2 | 15.6 | 26.2 |
| October 2003 | 4.5 | 19.6 | 25.3 |
| September 2006 | 3.9 | 23.5 | 32.6 |
| August 2007 | 4.3 | 28.7 | 40.7 |

He was put on low dose of a single stimulant medication. His norepinephrine level stayed normal. Dopamine and serotonin levels continued to increase and then normalized. It is again interesting to see that even though the dose was not altered much, these levels continued to be more and more normalized. His attention span and mood improved. School performance and self-esteem went up. He is currently in college and doing well. If the technology of the present invention was not used, most likely the physician would have increased his dose, giving him extra drug he did not need.

As exemplified by the above case, another embodiment of the present invention is to curtail criticism of using psychotropic drugs. The invention clarifies the chemical imbalance and algorithms guide the therapy based on the nature of chemical imbalance(s).

Chart 12 illustrates the use of present invention in a 27-year-old student. He presented with a history of depression and attentional difficulties. Table 7 shows his biomarker levels below:

TABLE 7

| Test date | NE | DA | 5HT |
|---|---|---|---|
| October 1999 | 2.3 | 11.9 | 9.6 |
| October 2000 | 4.1 | 16.3 | 27.2 |

His norepinephrine levels were normalized at the time of repeat testing. Dopamine levels improved thirty percent and serotonin hundred and seventy percent. He was able to continue his professional education. His dad wanted him to get SPECT scans. Thus he moved to the care of another clinic.

Another embodiment of the present invention is to evaluate and treat cases of substance abuse. To identify their exact chemical imbalance and treat it accordingly so they don't have to self-medicate and ‘go to the deep end.’

Chart 13 illustrates the use of the present invention in a 25-year-old female with a history of substance abuse. Before the treatment started, dopamine levels were abnormally high, see Table 8 below:

TABLE 8

| Test date | NE | DA | 5HT |
|---|---|---|---|
| June 2004 | 3.2 | 63.0 | 11.0 |
| November 2004 | 1.8 | 14.2 | 19.3 |

Norepinephrine was low and so was serotonin. She had a history of doing poorly in school, was socially odd and had exceptionally high level of energy. Impulsively she had made bad decisions in her life and currently was in a dysfunctional relationship. With treatment, substance abuse stopped. Her serotonin level increased seventy two percent in five months, dopamine level dropped from abnormal high to mildly low (normal range 17-30) and norepinephrine dropped by forty three percent. Due to her living out of the area, she discontinued treatment before all her neurotransmitter levels could be normalized.

Chart 14a illustrates the use of the present invention in an 11-year-old boy with ADHD and anxiety disorder. He presented with a history of declining school performance, despite trying, and self-consciousness. His levels are shown in Table 9 below:

TABLE 9

| Test date | NE | DA | 5HT |
|---|---|---|---|
| December 2002 | 1.4 | 7.3 | 19.8 |
| July 2003 | 1.9 | 18.3 | 22.6 |
| January 2004 | 1.9 | 20.5 | 23.3 |
| December 2004 | 2.8 | 22.3 | 33.9 |
| August 2005 | 2.9 | 25.7 | 40.7 |
| February 2007 | 3.7 | 30.5 | 61.2 |

He was put on one stimulant medication. The dose was quickly adjusted and then left the same for a while. Norepinephrine levels slowly started going up and in four some years they were close to lower normal. Dopamine levels went from being low to normal within less than a year and kept on going up at a slow and steady pace to end at near high normal. Serotonin also started as low and improved by 16 percent within a year. It normalized completely by Dec. '04 retesting, even though the dose and drug was kept the same.

Later in treatment, he wanted to gain weight and build muscles to impress girls in high school. So he was switched to an equivalent dose of similar drug, but different formulation, which did not suppress his appetite as much. He continues to do well at school. Serotonin levels have kept on increasing to much healthier levels, showing a two hundred percent increase within about four years. His anxiety has been in control, self-consciousness has improved and he is looking forward to start college.

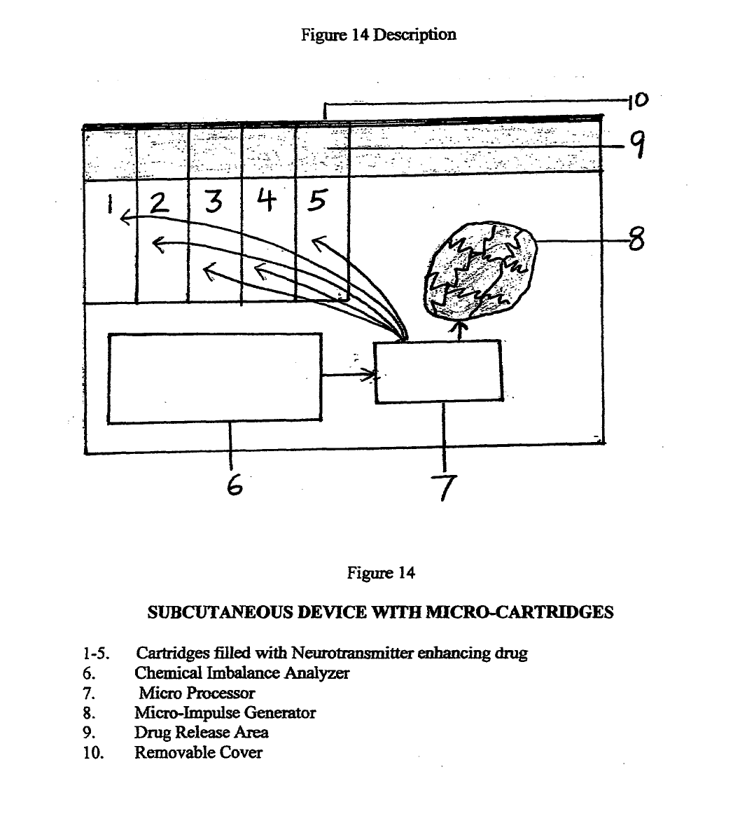

Chart 14b illustrates the use of the present invention in a 12-year-old boy, younger brother of the case in FIG. 14a. This youngster presented with low motivation, fidgetiness, being overweight and losing interest in school. His biomarker levels are shown in Table 10 below:

TABLE 10

| Test date | NE | DA | 5HT |
|---|---|---|---|
| April 2007 | 4.1 | 19.5 | 23.7 |
| November 2007 | 4.3 | 25.2 | 31.5 |

Previously, he had been prescribed a nor-epinephrine boosting drug by another respected physician. He did not like this drug. ‘It made me feel different,’ he had complained. There was no improvement in attentiveness or motivation. So he and his parents decided to discontinue the drug and see if he could pull himself together. The side effect of personality change went away but school difficulties continued. So he was brought to this clinic for a fresh evaluation.

Use of the present invention in this case, revealed norepinephrine and dopamine levels to be within normal limits. It was clear why he had responded negatively to the first drug. His norepinephrine levels were normal to begin with and he did not need a drug that would boost them further and create a new ‘norepinephrine imbalance,’ on top of his current problem, which was serotonin imbalance.

Based on the biomarker levels, the physician decided to put him first on a serotonin-boosting drug and see if his fidgetiness and attention improves. His biomarker levels were repeated in five months. Serotonin level had become normal. Clinically, he was not fidgety anymore. His mood improved and he did not complain of ‘feeling different.’

Despite improvement in fidgetiness and mood, his motivation and attention in school did not improve much. Since on repeat testing his dopamine level was in the mid-normal range, it was felt that it could be boosted and there was room for it to go up. So the physician decided to add a stimulant medication, different from the first one he had tried. And one that did not have much effect on the norepinephrine system. After couple of months on this drug, he still has not complained of ‘feeling different.’ He is doing better in school. Thus, the technology of the present invention has succeeded in treating him with no side effects. It has stopped the downward spiral of his self-esteem and saved his life.

Chart 15 illustrates the use of the present invention in a 36-year-old woman with history of depression, migraines and attentional difficulties. Her biomarker levels are shown in Table 11 below:

TABLE 11

| Test date | NE | DA | 5HT |
|---|---|---|---|
| September 1998 | 4.9 | 7.6 | 11.5 |
| October 1999 | 5.7 | 14.2 | 19.4 |

Biomarker evaluation showed she was struggling with low dopamine and low serotonin imbalance. Appropriate drug was used and the testing was repeated in a year. Dopamine levels improved by hundred percent and serotonin by seventy percent. Her depression and attention showed improvement and her migraines were improved. They were less frequent and less severe. Due to the present invention it was clear that her norepinephrine levels were normal, so no norepinephrine-boosting drug was used. These levels continued to stay normal at the time of repeat testing.

Chart 16 illustrates the use of the present invention in a 19-year-old young man. He presented with a history of not caring for his future, no sense of self-direction and lack of motivation. He came with his mother, father and step-dad. They all seem concerned about his poor performance in school. His biomarker levels are shown in Table 12 below:

TABLE 12

| Test date | NE | DA | 5HT |
|---|---|---|---|
| December 2000 | 1.9 | 18.9 | 27.1 |
| January 2003 | 3.2 | 21.6 | 25.5 |
| October 2004 | 4.0 | 25.6 | 28.2 |

His norepinephrine and serotonin levels were low and dopamine was low normal. Despite these results, he was adamant on not taking any drugs to correct them. ‘They will get better themselves, no big deal,’ was his attitude. Later he started to attend community college at an out of town location. Upon his first visit back home, he made an appointment to come to the clinic. He wanted to start treatment because school had been very difficult for him. His low attention span and easy distractibility had made him very frustrated. It was clear that he needed to do something about these issues or drop out of school. He was ready to correct his chemical imbalance.

He was started on a single norepinephrine-boosting type of drug. Norepinephrine level normalized by the time of repeat testing. Dopamine level improved to mid normal and serotonin improved to near low normal. He has been able to pay attention at his school. He continues to stay on the same drug he was prescribed initially and understands that his taking the drug is similar to ‘replacement treatment a myopic needs for his loss of distant vision by using correct eye-glasses.’ He has successfully completed college and made his parents proud.

Chart 17 illustrates the use of the present invention in a 39-year-old female. She presented with a history of high impulsivity, high level of energy, difficulties sustaining attention to detail and depression. Her biomarker levels are shown in Table 13 below:

TABLE 13

| Test date | NE | DA | 5HT |
|---|---|---|---|
| May 2001 | 1.7 | 6.8 | 14.9 |
| February 2004 | 3.4 | 12.8 | 20.5 |

Her norepinephrine, dopamine and serotonin levels were all low. She had a history of substance abuse as a youngster (No surprise all of her biomarkers were low). With treatment, her norepinephrine and dopamine each increased by hundred percent, respectively. Serotonin was about thirty percent higher, at the time of repeat evaluation, almost two and a half years later. It is interesting to note that her biomarker levels did not return to normal. The extent of her prior substance abuse in younger years may have caused some permanent damage. Had the technology of the current invention been used to help her at a younger age, self-medication and substance abuse may have been prevented.

Chart 18 illustrates the use of the present invention in a 44-year-old man who was referred by his wife for obsessive-compulsive problems, low-grade depression and attentional difficulties. Her complaints were,’ He obsessively hoards things and cannot throw them away. The house is a mess. He procrastinates.’ He was an obese gentleman, very intelligent, with a professional career. Every year he would overpay the IRS and was never organized enough to file his tax returns and get refund. His wife was angry and frustrated. His biomarker levels are shown in Table 14 below:

TABLE 14

| Test date | NE | DA | 5HT |
|---|---|---|---|
| November 2001 | 4.9 | 17.6 | 9.8 |
| April 2003 | 5.0 | 17.0 | 20.0 |

His norepinephrine levels were within normal limits. Dopamine was low normal and serotonin was extremely low. So he was put on a serotonin-boosting drug. He finally went for repeat testing about two and a half years later. There was no significant change in norepinephrine or dopamine levels. Intra-platelet serotonin levels increased by almost one hundred percent. His mood improved and anxiety lowered. He started working on his old tax returns and started filing them slowly. Due to downturn in the economy, he was laid off of work. That did not destabilize him. He diligently kept on looking for a new job and landed at a prestigious engineering job.

While at his new job, he started experiencing difficulties in concentration and distractibility. Looking at his biomarker profile, it was clear that his dopamine levels were low normal. Dopamine-boosting medication was suggested. He wanted to think about it. Due to family emergencies, he stopped coming. Treatment did help his obsessive-compulsive problems.

1. A composition comprising of a blood, urine, saliva and other biological cells and or fluids to diagnose chemical imbalance comprising of proteins, amines, peptides, amyloid, derivatives and or precursors, enzymes etc. related to molecules of Dopamine, Serotonin, Nor-epinephrine, epinephrine, Metanephrine, nor-Metanephrine, MAO enzyme, acetylcholine, glutamate, Gama amino Butyric acid, etc. to aid in diagnosis, guide therapy, prognosis and to determine pre-disposition of individuals to such mental and neurological illnesses where brain chemical imbalances of the neurotransmitters are involved.

2. The composition of claim 1, wherein such chemicals are measured in platelets cells and or other body cells, intracellularly.

3. A composition of claim 1, using software algorithm for managing medication and treatment of neuro-psychiatric patients, said algorithm comprising: Inputs for receiving concentrations of neurotransmitter levels in the biological fluids of said patient; Inputs for receiving patient mental status information; An algorithm that takes said concentrations of neurotransmitter levels and said patient mental status information as inputs, and produces drug and dosage recommendations and follow-up patient evaluation recommendations as outputs.

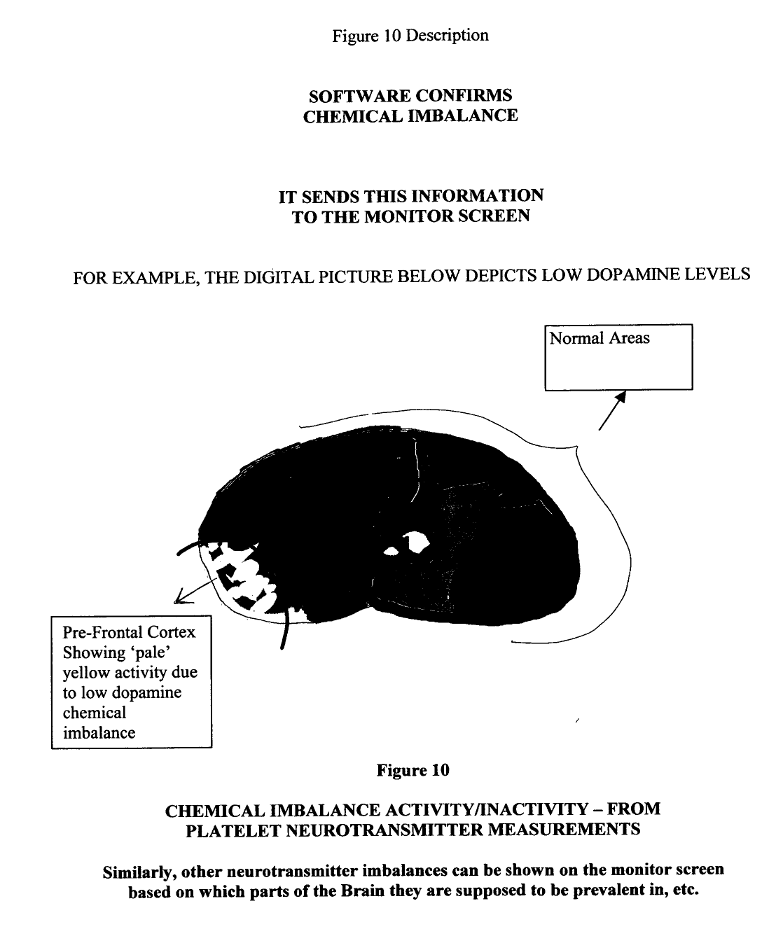

4. As illustrated in FIG. 10, the composition of claim 1, wherein measurements of neurotransmitter concentrations is used to portray colorful images and or images of the central nervous system on a computer screen identifying areas with adequate as well as inadequate concentrations of neurotransmitters. Also in other parts of the body where these neurotransmitters are playing a role in physiology and your pathology, for example stomach, adrenal glands, etc.

5. The composition of claim 1, wherein platelet measurements of neurotransmitters are used for diagnostic purposes, treatment planning and management of depression, A.D.H.D., bipolar disorder, autism, psychosis, schizophrenia, mania, learning disabilities, Parkinson's, Alzheimer's dementia, and other neuropsychiatric conditions.

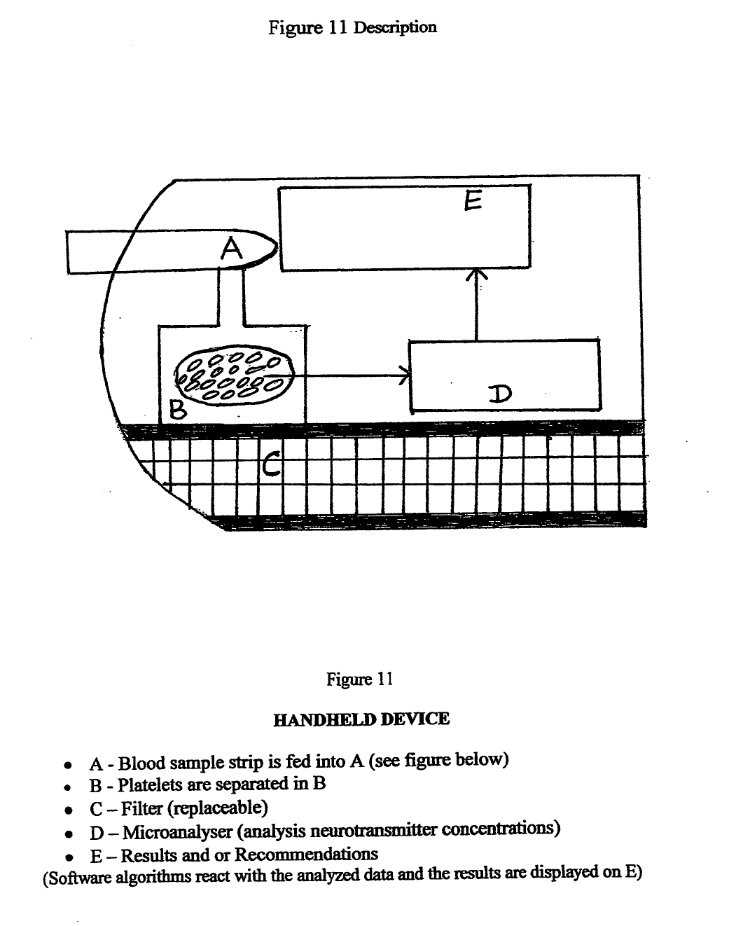

6. As illustrated in FIG. 11, the invention also entails test kit(s) which a customer can pick up at drugstore or superstore counters, like pregnancy test, etc. to measure platelet concentrations of single or multiple neuro transmitters, like Dopamine Serotonin would, Nor-epinephrine, acetylcholine, and others. These hand-held electronic computerized device(s), test kit(s) could also be used at doctor's offices. They may come with or without accompanying software, if the latter is already installed on the user's computer. Such testing to be used in humans and or in veterinary field

7. The composition of claim 1, wherein testing predicts risk factors for neuropsychiatric conditions-similar to a test that just came out which predicts if one is prone to developing baldness-even if the clinical symptoms have or have not yet emerged, and is also incorporated in the hand-held analyzers.

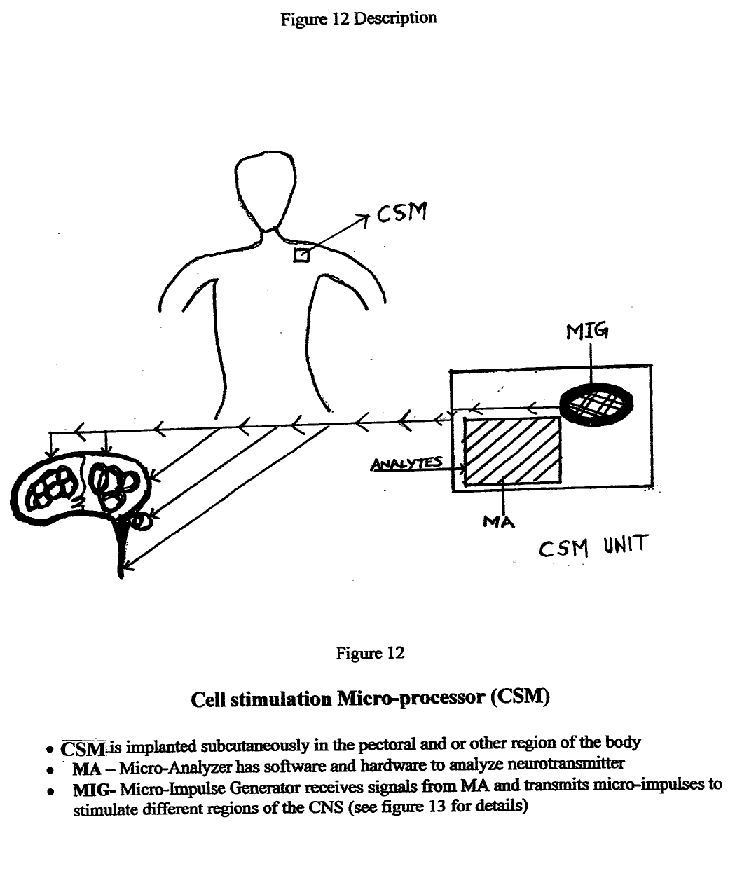

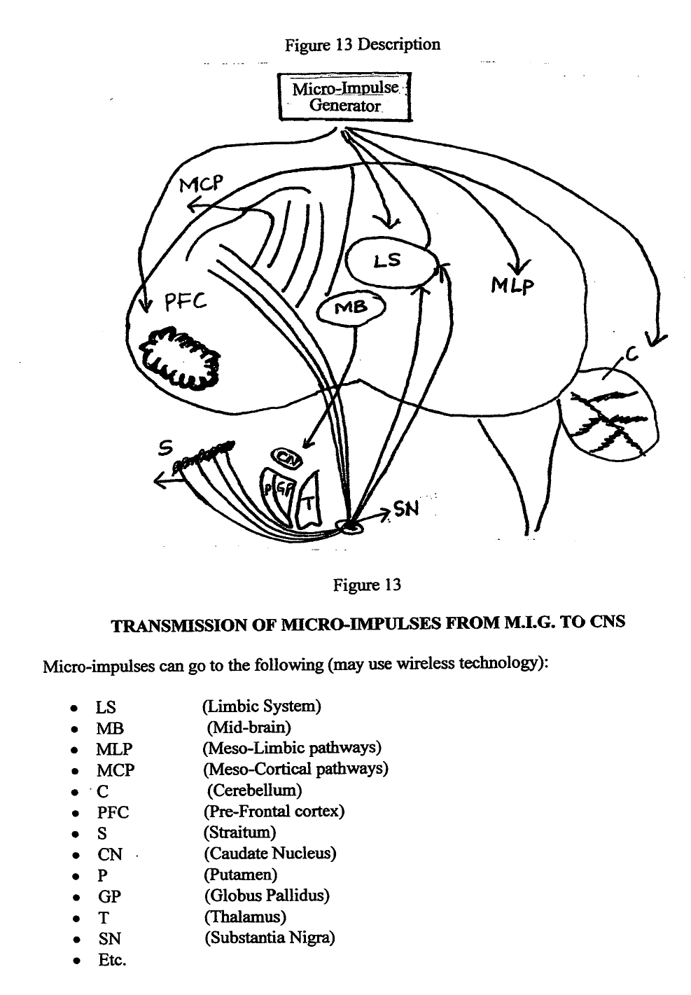

8. As illustrated in FIGS. 12 and 13—Cell stimulation device-a microchip on or under the skin and or muscles that at regular intervals keeps measuring these neurotransmitters concentrations and then stimulating cerebellum and or other parts of brain so chemicals are kept normalized every so many times in the day. This prevents the fluctuations in neurotransmitters in the central nervous system and other parts of the body and keeps symptoms of the various illnesses in control.

9. One of the embodiment of the invention entails the above device getting neurotransmitter data and analyzing it with software and transmitting it on wireless frequencies and other networks to doctor's office or offices and or call centers to monitor all the systems and validation.

10. As illustrated in FIG. 14, another embodiment of the invention is the above device in FIG. 12, filled with compartments of Serotonin, Dopamine, Nor-epinephrine, etc. producing and or stimulating compound or compounds. So whichever level is low the microprocessor chip squirts a calculated micro or milligrams of the compound. And, patients get indication before the compound and or the therapeutics solution is running out so that the cartridge is replaced at the doctor's office, etc. This way patients will not have to take multiple doses every day. The cartridge may be re-installed every three months or six months, etc.

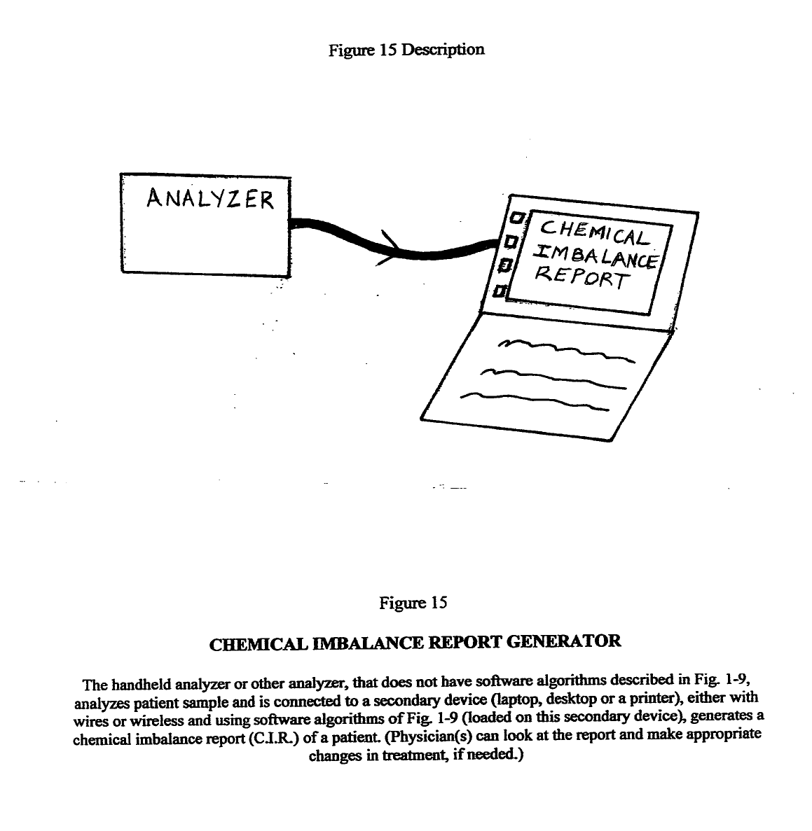

11. As illustrated in FIG. 15, another embodiment of the invention is a reader unit and or software that simplifies data of neurotransmitters and creates a report to guide in diagnosis and treatment, on a handheld, Laptop, Desktop and or any other similar computerized device.
This system which is not anticipated, rendered obvious, suggested, or even implied by any of the prior art, either alone or in any combination hereof. To attain this, one of the main components of this invention is the software that makes the data on chemical imbalance easily readable. Its algorithms can point treatment steps to the primary care physician, physician assistants and other health professionals and guide in follow-up strategies.
There has thus been outlined, rather broadly, the more important features of the invention in order that the detailed description thereof may be better understood, and in order that the present contribution to prior art may be better appreciated. There are additional features of the invention that can be described hereinafter. In this respect, before explaining at least one embodiment of the invention in detail, it is to be understood that the invention is not limited in its application to the details of construction and to the arrangements of the compliments set forth in the following description or illustrated in the drawings. The invention is capable of other embodiments and of being practiced and carried out in various ways. Also, it is to be understood that the phraseology and terminology employed herein are for the purposes of description and should not be regarded as limiting. To the accomplishment of the above and related objects, this invention may be embodied in the form illustrated in the accompanied drawings, attention being accorded to the fact, however, that the drawings are illustrative only, and that changes may be made in the specific construction illustrated.
